(19) United States
(12) Patent Application Publication
(10) Pub. No.: US 2016/0175578 A1
(43) Pub. Date: Jun. 23, 2016

(54) DIRECTIONAL CONTROL VALVE FOR USE IN CATARACT SURGERY

(71) Applicant: Episonic, LLC, North Canton, OH (US)

(72) Inventor: Philip C. ROHOLT, Canton, OH (US)

(21) Appl. No.: 14/960,571

(22) Filed: Dec. 7, 2015

Related U.S. Application Data
(60) Provisional application No. 62/087,902, filed on Dec. 5, 2014.

(51) Int. Cl. A61M 39/22; A61M 1/00; A61F 9/007

(52) U.S. Cl. CPC A61M 39/223; A61F 9/00745; A61M 1/0084

(57) ABSTRACT

A directional control valve for use in cataract surgery typically includes a valve, body and a valve switch. The valve body includes ports for inflow and outflow of fluid, as well as a hollowed out portion in which a core portion of the valve switch sits. The core portion of the valve switch can be in the form of a circular disc having internal core ports which can be caused to reversibly rotate between a first position and a second position, thereby causing fluid flow to the inflow/outflow ports to be alternately opened and closed, depending on which position the valve switch is turned.

### CROSS-REFERENCE TO RELATED APPLICATION

This application claims the benefit of U.S. Provisional Application No. 62/087,902, filed on Dec. 5, 2014, the disclosure of which is incorporated herein by reference in its entirety.

### FIELD OF THE INVENTION

The present invention relates generally to valves useful in medical devices, and more particularly to a directional control valve for cataract surgery.

### BACKGROUND OF THE INVENTION

The use of ultrasonic instruments in surgical applications is well known. Phacoemulsification cataract surgery is a procedure in which an ultrasonic device such as a phacoemulsifier handpiece is used to break up and then remove a cloudy lens, or cataract, from the eye to improve vision. The insertion of an intraocular lens (IOL) usually immediately follows phacoemulsification. During phacoemulsification surgery (or simply “phaco”, as surgeons refer to it) the patient's natural lens is ultrasonically emulsified in situ through the use of a vibrating needle. Piezoelectric crystals are typically utilized to vibrate the needle, and a saline solution is generally infused between an outside of the needle and a flexible shield in order to both cool the needle and irrigate the exposed eye cavity. The emulsified lens is then aspirated from the eye through the needle lumen, with the aid of continued infusion/irrigation of saline solution.

During a typical Phaco procedure the surgeon uses two separate instruments in consecutive fashion, i.e. a phacoemulsifier handpiece and an irrigation/aspiration (I/A) handpiece. Specifically, once the nucleus of the lens has been ground up and aspirated by the phaco handpiece, an I/A handpiece is used which does not have a vibrating needle but an inner tube designed to provide for aspiration. The I/A handpiece is typically rounded at the end and believed to be safer for final removal of the relatively soft cortical material or cortex which surrounds the hard nucleus of a lens. Both of these handpieces typically have inflow and outflow fittings which share the same input and output tubing, so that when the surgeon wants to switch from using the phaco to the I/A handpiece, typically the two tubes connected to the phaco handpiece are detached by an assistant and then connected to the I/A handpiece. This process can cause an interruption in the course of a delicate cataract surgery, causing problems for a variety of reasons.

For example, in haste to quickly switch from one handpiece to the other, the tubing connections may not be complete or sufficient, resulting in detachment and loss of inflow or outflow control. This can cause collapse or rupture of the anterior chamber of the eye, with ensuing complications such as posterior capsule tear, or damage to the corneal endothelium. Sometimes the tubing connections can get stuck during transition, requiring, the use of a hemostat or the like to achieve removal of the tubing. There are also instances when the surgeon needs to go back to the first handpiece to further break down the lens, and the connections need to be redone. Repeated manual reconnection of the tubing thus can be problematic.

In light of the above, it would be advantageous to provide more precise control of ultrasonic instruments in surgical applications such as cataract surgery and other ophthalmological procedures, particularly during such steps as ultrasound application, phacoemulsification, or aspiration and irrigation of fluids from bodily cavities. It would therefore be desirable to provide an improved surgical valve device wherein the tubing connections to both the ultrasound device and the I/A handpiece are separate from one another. It would also be advantageous to eliminate the need for an assistant to manually switch the tubing from one handpiece to another. It would also be advantageous to provide a directional control valve that allows the cataract surgeon to immediately and seamlessly switch between the two devices as needed.

### SUMMARY OF THE INVENTION

The directional control valve device of the present invention solves the problems noted above; all the tubing connections for both the phaco and I/A handpiece can be quickly and easily completed prior to the start of the surgery, with no switching needed.

A first aspect of the invention provides a directional control valve for alternately connecting to a phacoemulsification handpiece and an irrigation/aspiration handpiece during cataract surgery, the control valve comprising a valve body and a valve switch, wherein the valve switch is reversibly rotatable between a first position and a second position.

A second aspect of the invention provides a directional control valve for alternately connecting to a phacoemulsification handpiece and an irrigation/aspiration handpiece during cataract surgery, the control valve comprising a valve body comprising a hollowed out internal portion and an external portion and a valve switch comprising a peripheral portion for reversibly rotating the valve switch between a first position and a second position and a core portion seated within the hollowed out internal portion of the valve body and connected to the peripheral portion of the valve switch for being reversibly rotated between the first position and the second position.

A third aspect of the invention provides a directional control valve for alternately connecting to a phacoemulsification handpiece and an irrigation/aspiration handpiece during cataract surgery, the control valve comprising a valve body comprising a hollowed out internal portion and an external portion, the external portion comprising a proximal side having a proximal inflow port and a proximal outflow port and a distal side having a first distal outflow port, a second distal outflow port, a first distal inflow port and a second distal inflow port, and a valve switch comprising a peripheral portion for reversibly rotating the valve switch between a first position and a second position and a core portion seated within the hollowed out internal portion of the valve body and connected to the peripheral portion of the valve switch for being reversibly rotated between the first position and the second position, the control valve being alternately connectable to a phacoemulsification handpiece in the first position and to an irrigation/aspiration handpiece in the second position, the core portion including a first core port and a second core port, wherein the first core port aligns with the proximal inflow port and the first distal outflow port in the first position, the second core port aligns with the proximal outflow port and the first distal inflow port in the first position, the first core port aligns with the proximal inflow port and the second distal outflow port in the second position, and the second core port aligns with the proximal outflow port and the second distal inflow port in the second position. The nature and advantages of the present invention will be more fully appreciated after reviewing the accompanying drawings, detailed description and claims.

### BRIEF DESCRIPTION OF THE DRAWINGS

The accompanying drawings illustrate embodiments of the invention and, together with a general description of the invention given above, and the detailed description given below, serve to explain the principles of the invention.

FIG. 1 is a top plan view depicting tubing for connecting a phaco handpiece to an exemplary control valve of the present invention;

FIG. 2 is a top plan view depicting tubing for connecting an Irrigation/Aspiration device to the control valve of FIG. 1;

FIG. 3 is a front view of the control valve of FIG. 1;

FIG. 4 is a rear view of the control valve of FIG. 1;

FIG. 5 is a side view of the control valve of FIG. 1.

### DETAILED DESCRIPTION OF THE INVENTION

The directional control valve of the present invention allows disposable surgical tubing connections for both a phaco device and a subsequently-used I/A device to be quickly and easily completed prior to the start of the surgery. When the surgeon has completed the phaco portion of the cataract surgery and is ready to use the second instrument, the assistant can simply turn a switch on the inventive valve device and the I/A hand piece is immediately available. If the surgeon subsequently needs to return to the phaco handpiece, placing the valve switch to the original position allows the phaco instrument to be immediately available. The inventive device thus provides an improvement over prior practice which can involve problems associated with manually switching/reconnecting between device tubing each time.

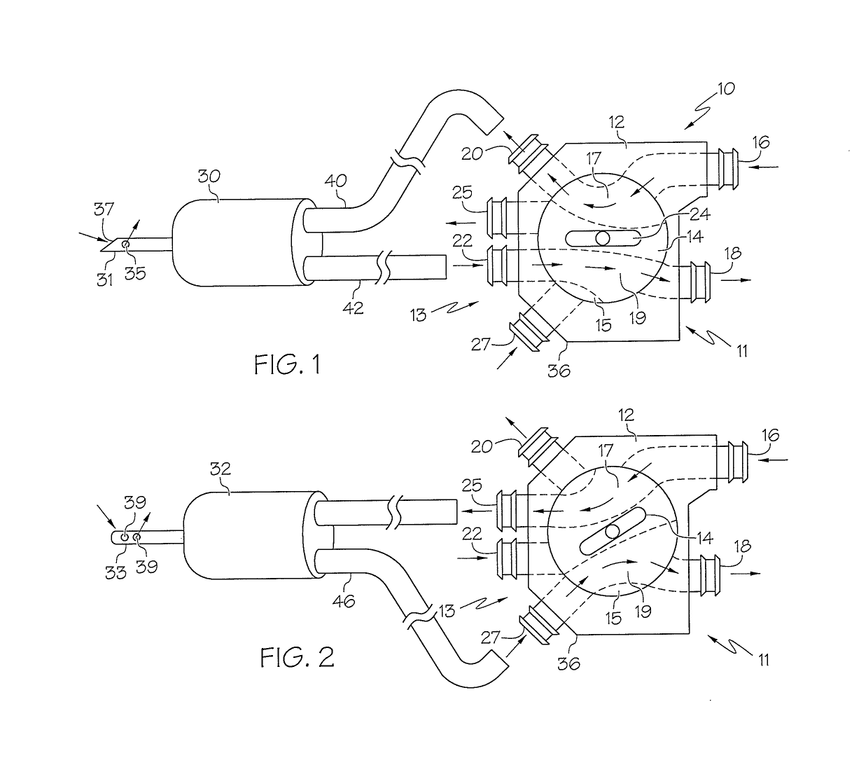

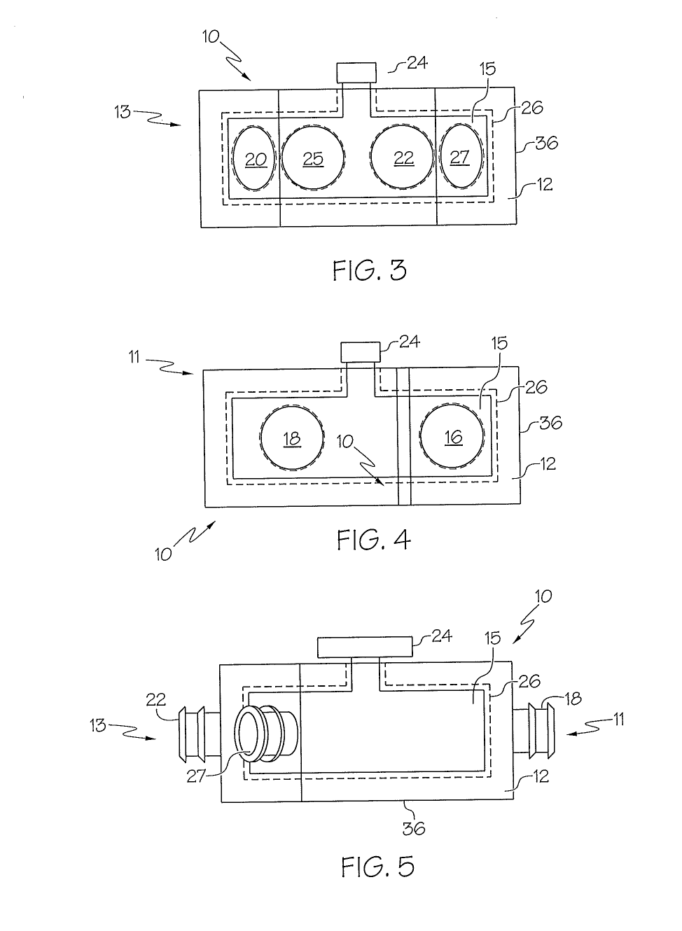

FIGS. 1-5 show an exemplary control valve 10 according to the present invention. Each valve 10 includes valve body 12 and a valve switch 14. The valve body 12 includes a hollowed out internal portion 26 and an external portion 36 (best seen in FIG. 5). The external portion 36 has a proximal side 11 which includes a proximal inflow port 16 and a proximal outflow port 18, and a distal side 13 which includes a first distal outflow port 20, a second distal outflow port 25, a first distal inflow port 22, and a second distal inflow port 27.

The valve switch 14 includes a core portion 15 that is typically rotatably seated within the hollowed out internal portion 26 of the valve body 12. The core portion 15 can be in the form of a circular disc having an external geometry that corresponds to the size and shape of the hollowed out internal portion 26 of the valve body 12. The core portion 15 also includes an internal geometry that forms a first core port 17 and a second core port 19. The core portion 15 is connected to and thus rotatable upon movement of a peripheral portion 24 of the valve switch 14 by a user. Specifically, the peripheral portion 24 of the valve switch 14 can be grasped and moved by a user causing the core portion 15 to reversibly rotate between a first position (shown in FIG. 1) and a second position (shown in FIG. 2). As the valve switch 14 is moved/rotated between first and second positions, fluid flow to the first distal outflow port 20, the second distal outflow port 25, the first distal inflow port 22, and the second distal inflow port 27 is alternately opened and closed, depending on the position that the switch 14 is turned to.

Specifically, in the first position the first core port 17 aligns with the proximal inflow port 16 and the first distal outflow port 20, thereby allowing fluid to flow from the proximal inflow port 16, through the first core port 17, and then out of the device through the first distal outflow port 20 (but not through the second distal outflow port 25). Likewise, in the first position the second core port 19 aligns with the proximal outflow port 18 and the first distal inflow port 22, thereby allowing fluid to flow from the first distal inflow port 22 (but not from the second distal inflow port 27) through the second core port 19, and out of the device through the proximal outflow port 18.

In the second position, the first core port 17 aligns with the proximal inflow port 16 and the second distal outflow port 25, thereby allowing fluid to flow from the proximal inflow port 16, through the first core port 17, and then out of the device through the second distal outflow port 25 (but not through the first distal outflow port 20). Likewise, in the second position the second core port 19 aligns with the proximal outflow port 18 and the second distal inflow port 27, thereby allowing fluid to flow from the second distal inflow port 27 (but not from the first distal inflow port 22) through the second core port 19, and out of the device through the proximal outflow port 18. It can be appreciated that in both the first position and the second position the core ports 17 and 19 always allow fluid to flow through the proximal inflow port 16 and the proximal outflow port 18 at the proximal side 11 of the valve body 12, no matter which position the switch 14 is turned to. This is best appreciated by viewing the directional flow arrows in FIGS. 1 and 2.

FIGS. 1 and 2 illustrate the valve 10 alternately connected to a phacoemulsification handpiece 30 and an irrigation/aspiration handpiece 32. Each of the handpieces 30, 32 have at least one and typically a plurality of openings 35, 39, through which the irrigation liquid can flow to enter and/or exit the operating field. Looking at FIG. 1, the phaco handpiece 30 is connected to port 20 of the inventive valve 10 via tubing 40, as well as to port 22 via tubing 42. As shown in FIG. 2. the I/A handpiece 32 is connected to port 25 via tubing 44, and to port 27 via tubing 46. It is noted that, in actual use, both handpieces 30 and 32 and their associated disposable surgical tubing 40, 42, 44, 46 are intended to be connected to the inventive valve 10 at the same time. The division of this arrangement into separate FIGS. 1 and 2 is merely to provide a more clear understanding of the invention. The valve 10 includes a valve body 12 and a valve switch 14 for alternately switching between the two handpieces 30, 32. The valve switch typically includes a peripheral, external portion 14 connected to an internal core portion 15 which is rotatably seated within the hollowed out internal portion 26 of the valve body 12.

In use, tubing typically leads from a hanging saline bag (not shown) to create an inflow circuit supplying fluid in the direction of the arrow leading to port 16. This inflow circuit is connected to the proximal inflow port 16 of external portion 36 of the valve body 12. In the arrangement shown in FIG. 1 in which the switch 14 is in the first position, the first distal outflow port 20 and first distal inflow port 22 are “open” and the second distal outflow port 25 and second distal inflow port 27 are “closed”. That is, in the first position the core portion 15 of the valve switch 14 allows saline flowing in through the proximal inflow port 16 to pass through the first core port 17 and out through the first distal outflow port 20. From there, irrigation fluid flows through tubing 40 to the phaco handpiece 30, and then out through opening 35 located in the tip 31 of the phaco handpiece. Upon aspiration by the surgeon, fluid can then be caused to exit the surgical field and enter the phaco handpiece via opening 37 in the tip 31 of the phaco handpiece 30, and then consecutively pass through tubing 42, the first distal inflow port 22 of the valve body, the second core port 19, and then exit through the proximal outflow port 18, which is typically connected to tubing (not shown) leading to a drainage reservoir or waste bin.

In the arrangement shown in FIG. 2 in which the switch 14 has been moved/rotated to the second position, first distal outflow port 20 and first distal inflow port 22 are “closed” and the second distal outflow port 25 and second distal inflow port 27 are “open”. That is, in the second position the core portion 15 of the switch allows saline flowing in through proximal inflow port 16 to pass through the first core port 17 and out through the second distal outflow port 25. From there, irrigation fluid flows through tubing 44 to the handpiece 32, and then out through openings 39 located in the tip 33 of the I/A handpiece 32. Upon aspiration by the surgeon, fluid can then be caused to exit the surgical field and enter the I/A handpiece via openings 39 in the tip 31 of the I/A handpiece 32, and then consecutively pass through tubing 46, the second distal inflow port 27, the second core port 19 and then exit through the proximal outflow port 18.

As can be appreciated from the above description and from viewing the flow arrows of FIG. 1 and FIG. 2, core portion 15 of the valve switch 14 can be manufactured in the form of a circular disc having an internal geometry which allows its first and second core ports 17 and 19 to alternately open and close fluid flow to ports 20, 22, 25 and 27, depending on the position that the switch 14 is turned to, while ports 17 and 19 always allow fluid to flow through ports 16 and 18 of the valve. In this arrangement, i.e. in which the tubing 40, 42 for the phaco handpiece 30 and the tubing 44, 46 for the I/A handpiece 32 are separate from one another, the surgeon is able to immediately and seamlessly switch between the two handpieces 30, 32 as needed with a turn of the peripheral portion 24 of the valve switch 14 from the first position to the second position, eliminating the need for an assistant to manually switch the tubing from one handpiece to another. The tubing connections to the various inflow/outflow ports can be completed by a surgical assistant or the like in advance of the surgical procedure.

The valve device of the present invention can be manufactured as a single-use, disposable device, with the valve body 12 and valve switch 14 portions made of a plastic or thermoplastic material such as polypropylene, polyoxymethylene, polyformaldehyde or the like. O-rings can be included to seal against fluid leakage, as is known in the art. The device can also be manufactured as a multiple-use, autoclaveable stainless steel device. Such methods of production are well known to those familiar with the art.

For example, the original tubing emanating from a commercial Phaco-I/A cassette can be cut and attached to the ports of the valve. The tubing connected to the phacoemulsifier hand piece is then connected to the appropriate in/out of the valve. The other in/out lines from the valve (provided) are then connected to the I/A handpiece. Alternatively, a disposable, single use device from Baush & Lomb (e.g. the 85910ST Silicone I/A Handpiece for the Stellaris® System or the 85910S Silicone I/A Handpiece for the Millennium™) could come pre-attached to the control valve with its own dedicated tubing.

While the present invention has been illustrated by the description of embodiments thereof in considerable detail, it is not intended to restrict or limit the scope of the appended claims to such detail. Additional advantages and modifications will be readily apparent to those skilled in the art. Accordingly, departures may be made from such details without departing from the scope of the invention.

What is claimed is:

1. A directional control valve for alternately connecting to a phacoemulsification handpiece and an irrigation/aspiration handpiece during cataract surgery, the control valve comprising:
a) a valve body; and
b) a valve switch, wherein the valve switch is reversibly rotatable between a first position and a second position.

2. The directional control valve of claim 1, wherein the control valve is alternately connectable to a phacoemulsification handpiece in the first position and to an irrigation/aspiration handpiece in the second position.

3. The directional control valve of claim 1, the valve body comprising:
i) a hollowed out internal portion; and
ii) an external portion,
the valve switch comprising:
i) a peripheral portion; and
ii) a core portion, wherein the core portion is seated within the hollowed out internal portion of the valve body and connected to the peripheral portion of the valve switch for being reversibly rotated between the first position and the second position.

4. The directional control valve of claim 3, the external portion of the valve body comprising:
i) a proximal side having a proximal inflow port and a proximal outflow port; and
ii) a distal side having a first distal outflow port, a second distal outflow port, a first distal inflow port and a second distal inflow port.

5. The directional control valve of claim 4, the core portion comprising:
i) a first core port; and
ii) a second core port, wherein the first core port aligns with the proximal inflow port and the first distal outflow port in the first position, the second core port aligns with the proximal outflow port and the first distal inflow port in the first position, the first core port aligns with the proximal inflow port and the second distal outflow port in the second position, and the second core port aligns with the proximal outflow port and the second distal inflow port in the second position.

6. A directional control valve for alternately connecting to a phacoemulsification handpiece and an irrigation/aspiration handpiece during cataract surgery, the control valve comprising:
a) a valve body comprising:
i) a hollowed out internal portion; and
ii) an external portion; and
b) a valve switch comprising:
i) a peripheral portion for reversibly rotating the valve switch between a first position and a second position; and
ii) a core portion seated within the hollowed out internal portion of the valve body and connected to the peripheral portion of the valve switch for being reversibly rotated between the first position and the second position.

7. The directional control valve of claim 6, wherein the control valve is alternately connectable to a phacoemulsification handpiece in the first position and to an irrigation/aspiration handpiece in the second position.

8. The directional control valve of claim 6, wherein the external portion of the valve body comprises a proximal side having a proximal inflow port and a proximal outflow port, and a distal side having a first distal outflow port, a second distal outflow port, a first distal inflow port and a second distal inflow port.

9. The directional control valve of claim 8, the core portion including a first core port and a second core port, wherein the first core port aligns with the proximal inflow port and the first distal outflow port in the first position and with the proximal inflow port and the second distal outflow port in the second position, and wherein the second core port aligns with the proximal outflow port and the first distal inflow port in the first position and with the proximal outflow port and the second distal inflow port in the second position.

10. A directional control valve for alternately connecting to a phacoemulsification handpiece and an irrigation/aspiration handpiece during cataract surgery, the control valve comprising:
a) a valve body comprising:
i) a hollowed out internal portion; and
ii) an external portion, the external portion comprising a proximal side having a proximal inflow port and a proximal outflow port, and a distal side having a first distal outflow port, a second distal outflow port, a first distal inflow port and a second distal inflow port; and
b) a valve switch comprising:
i) a peripheral portion for reversibly rotating the valve switch between a first position and a second position; and
ii) a core portion seated within the hollowed out internal portion of the valve body and connected to the peripheral portion of the valve switch for being reversibly rotated between the first position and the second position,
the control valve being alternately connectable to a phacoemulsification handpiece in the first position and to an irrigation/aspiration handpiece in the second position, the core portion including a first core port and a second core port, wherein the first core port aligns with the proximal inflow port and the first distal outflow port in the first position, the second core port aligns with the proximal outflow port and the first distal inflow port in the first position, the first core port aligns with the proximal inflow port and the second distal outflow port in the second position, and the second core port aligns with the proximal outflow port and the second distal inflow port in the second position.
